(19) United States
(12) Patent Application Publication
(10) Pub. No.: US 2002/0182616 A1
(43) Pub. Date: Dec. 5, 2002

(54) Single nucleotide polymorphisms

(76) Inventors: Claes Wahlestedt, Stockholm (SE); Bo Ding, Sundbyberg (SE)

Correspondence Address: DINSMORE & SHOHL, LLP, 1900 CHEMED CENTER, 255 EAST FIFTH STREET, CINCINNATI, OH, 45202, US

(21) Appl. No.: 10/002,048

(22) Filed: Nov. 2, 2001

Related U.S. Application Data
(60) Provisional application No. 60/249,976, filed on Nov. 20, 2000.

(30) Foreign Application Priority Data
Nov. 3, 2000 (SE) 0004035-2

(51) Int. Cl. C12Q 1/68

(52) U.S. Cl. 435/6

(57) ABSTRACT

The present invention relates to the identification of common single nucleotide polymorphisms (SNPs) in the human neuropeptide Y (NPY) gene. The invention also relates to methods for diagnosis of genetic susceptibility for obesity, based on the association with increased body-mass (BMI) of a leucine(7)-to-proline(7) polymorphism in the NPY signal peptide.

### TECHNICAL FIELD

The present invention relates to the identification of common single nucleotide polymorphisms (SNPs) in the human neuropeptide Y (NPY) gene. The invention also relates to methods for diagnosis of genetic susceptibility for obesity, based on the association with increased body-mass index (BMI) of a leucine(7)-to-proline(7) polymorphism in the NPY signal peptide

### BACKGROUND ART

Obesity causes many health problems, both independently and in association with other diseases. In clinical practice, body fatness is assessed by Body-Mass Index (BMI) which is defined as weight in kilograms divided by square of the height in meters. A World Health Organzation (WHO) expert committee has proposed the classification of overweight and obesity and defines obesity as a BMI above 30 kg/m². Many studies have indicated that obesity is a highly heritable trait, with genetic variation estimated to account for 40-70% of the interindividual variation in body mass (Stunkard et al, 1986, N. Eng. J. Med. 314:193-9). Studying rare mutations in humans provides fundamental insight into a complex physiological process, and complements population-based studies that seek to reveal primary causes. In Sweden, although the prevalence of obesity is lower than in the United States, it is increasing at an alarming rate, especially in children (World Health Organization. Geneva, 1998, Obesity: Preventing and managing the global epidemic). There is consequently a need for the identification of genes that predispose an individual to obesity

Single nucleotide polymorphism (SNP) is single base pair position in genomic DNA at which different sequence alternatives (alleles) exist in normal individuals across populations. SNP studies are known to be an approach to survey human genetic variation at the DNA level (see eg Campbell et al. (2000) Drug Discovery Today 5; 388-396). Discovery and scoring of SNPs or combinations of SNPs (haplotype analysis) can, for example, be of importance to understand obesity mechanisms and provide possibilities towards better treatment. Numerous methods exist for the detection of SNPs within a nucleotide sequence (for a review, see Landegren et al (1998) Genome Res. 8: 769-776).

Neuropeptide Y (NPY) is a well-studied 36 amino acid neuromodulator that is secreted by neurons in the central and peripheral nervous system and is the most abundant and widely distributed of neuropeptide discovered to date. Since its discovery in 1982 (Tatemoto et at. (1982) Nature 296: 659-660), NPY has been shown to play a critical role in the regulation of satiety, reproduction, the central endocrine, cardiovascular systems and many other physiological processes, such as potent stimulation of food intake and associated weight gain in animal models (Wahlestedt, C. and Reis. D. J. (1993) Annu. Rev. Pharmacol. Toxicol. 32: 309-352.).

The human NPY gene is located on chromosome 7q15.1 and is about 8 kilobases in length with four exons separated by three introns of approximately 965, 4300 and 2300 bp. The gene (represented by the cDNA sequence set forth as SEQ ID NO: 1) produces a precursor protein (pre-pro-NPY; SEQ ID NO: 2) that includes a signal peptide (amino acids 1-28 in SEQ ID NO: 2), mature NPY (amino acids 29-64 in SEQ ID NO: 2), and a carboxyl-terminal flanking peptide with no known function (amino acids 65-97 in SEQ ID NO: 2) (Minth et al. (1984) Proc. Natl. Acad. Sci. U.S.A. 81: 4577-4581; GenBank Accession No. K01911).

Four segments of the human NPY genomic nucleotide sequence (Minth et al. (1986) J. Biol. Chem. 261:11974-11979, GenBank Accession Nos. M14295; M14296; M14297; and M14298) are shown as SEQ ID NOS: 3. 6. The nucleotide sequence encoding pre- pro-NPY is obtained by joining the exons shown as positions 30-217 in SEQ ID NO: 4, positions 31-111 in SEQ ID NO: 5; and positions 32-56 in SEQ ID NO: 6. The complementary sequence of the human NPY gene is also comprised in the genomic sequence published with GenBank Accession No. AC004485. The coding sequence is obtained by joining positions 22780-22804, 24888-24968; and 29039-29226 of AC004485.

This polymorphism was not associated with obesity or energy metabolism, but was significantly associated with high serum total and LDL cholesterol levels

Recent association studies showed that a T1128C (leucine by proline at residue 7 in the signal peptide part of pre-pro-NPY) polymorphism is associated with high serum cholesterol, LDL cholesterol levels both in normal-weight and obese Finns and in obese Dutch subjects (Karvonen et al. 1998, Nature Medicine 4: 1434-1437), enhanced carotid atherosclerosis in elderly patients with type 2 diabetes and control subjects (L Niskanen et al., J Clin Endocrinol Metab 85, 2266 (2000)), retinopathy in type 2 diabetes (L. Niskanert et al., Exp Clin Endocrinol Diabetes 108, 235 (2000)), birth weight and serum triglyceride concentration in preschool aged children (M. K. Karvonen et al., J Clin Endocrinol Metab. 85,1455(2000).

Kauhanen et al. (2000, Am. J. Med. Genet 93: 117-121) analyzed 889 middle-aged men from eastern Finland for the leucine(7)-to-proline(7) polymorphism of NPY. The gene variant producing the Pro(7) substitution was associated with a 34% higher average alcohol consumption. The authors suggested that alcohol preference in humans might be regulated by the NPY system.

A -880 2 bp I/D promoter region variant is associated with body mass and fat patterning in non-obese Mexican Americans (M. B. Bray, E. Boerwinkle, C. L. Hanis, Obes. Res. 8,219 (2000)).

WO 00/63430 (Hormos Medical Oy Ltd.) discloses methods for diagnosing a person's susceptibility for having an increased risk for the development of atherosclerosis, or of diabetic retinopathy. The methods are based on leucine(7)-to-proline(7) polymorphism in the pre-pro neuropeptide Y gene.

### BRIEF DESCRIPTION OF THE DRAWINGS

FIG. 1 SNPs observed within the NPY promoter and coding region. Base positions are based on the NPY start sites as identified by Minth et al., 1986 (cf. FIG. 7).

FIG. 2 Frequency distribution of BMI values in (A) POLCA population, and (B) OBESITY population.

FIGS. 3A and 3B Box plots showing BMI (kg/m²) of individuals with 1128T/T and 1128T/C genotypes, respectively, in (A) POLCA population (n-572), and (B) OBESITY population (BMI below 30 kg/m²) (n=396).

FIGS. 4A and B Proposed models of the three dimensional structure of (a) wild type signal peptide residues 1-14 MLGNKRLGLSGLTL) and (b) the Leu 7Pro signal peptide residues 1-14 (MLGNKRPGLSGLTL

FIGS. 5A and 5B Effect of single co-injection of NPY with and without and wild signal peptides on food intake post injection of the peptides

FIG. 6 Translocation in the mutants compared with wild-type NPY. Abbreviations: wt, wild-type; mut, mutant; NLT, acceptor peptide for oligosaccharyl transferase (inhibiting glycosylation); signal peptide, signal peptide plus P2 domain; signal peptide plus NPY, full-length NPY.

FIG. 7 Nucleotide sequence of the human NPY gene (Adapted from Minth et al. (1986) J. Biol. Chem. 261, 11974-11979). Capital letters indicate exons and lower case letters are used for introns and flanking sequences. +1 indicates the start site for the primary transcript. The sequence coding for pre-pro-NPY is underlined. IVS, intervening sequence. Nucleotide changes in positions 1258 and 5671 have previously been identified by Minth et al., supra, and the change in position 1128 by Karvonen et al., supra

### DISCLOSURE OF THE INVENTION

We have data supporting the finding that the 1128 polymorphism is associated with increased body weight in otherwise normal and healthy adult Swedish men and women.

We can offer an explanation why it is that the 1128 polymorphism (which gives rise to a amino acid change, leucine to proline) causes increased body mass index (BMI). The amino acid change gives rise to an altered signal peptide that in itself is able to augment the stimulatory effect of neuropeptide Y (NPY) on food intake in rats.

We also show that the polymorphism does not cause altered processing of preproneuropeptide Y; our data sharply disagree thus with those of Kallio et al. 2001 (FASEB J. 15(7):1242-4).

We suggest that this phenomenon can also occur in humans and therefore account for the increase in BMI (an indicator of obesity) that is seen in humans with this polymorphism.

These findings are of utmost importance for diagnosis and possible predisposition of obesity.

In order to identify genes that predispose an individual to obesity, the inventors have studied (i) to what extent the human NPY gene (coding and promoter regions) is polymorphic in the Swedish population and (ii) whether such polymorphism might contribute to phenotypic parameters related to obesity.

This invention is thus based on the identification, in 30 healthy Swedish individuals, of ten identified common human sequence variations (single nucleotide polymorphisms, SNPs) within the regulatory and coding sequences of the human NPY gene. Five of the SNPs were used to genotype two large Swedish cohorts of individuals. One variant, a leucine(7)-to-proline(7) polymorphism in tile signal peptide of NPY was found to be associated with body mass index (BMI) in both cohorts. However, in vitro translocation studies suggest that the polymorphism in the signal peptide region does not affect the site of cleavage and targeting or uptake of NPY into the endoplasmic reticulum (ER). While NPY is well known to potently stimulate food intake in experimental animals, we here show the first data that link an alteration in the human NPY signaling system to human body weight.

Consequently, in a first aspect this invention provides a method for diagnosing predisposition for obesity in a human individual, comprising

(a) obtaining a biological sample containing at least one nucleic acid molecule from said human individual; and

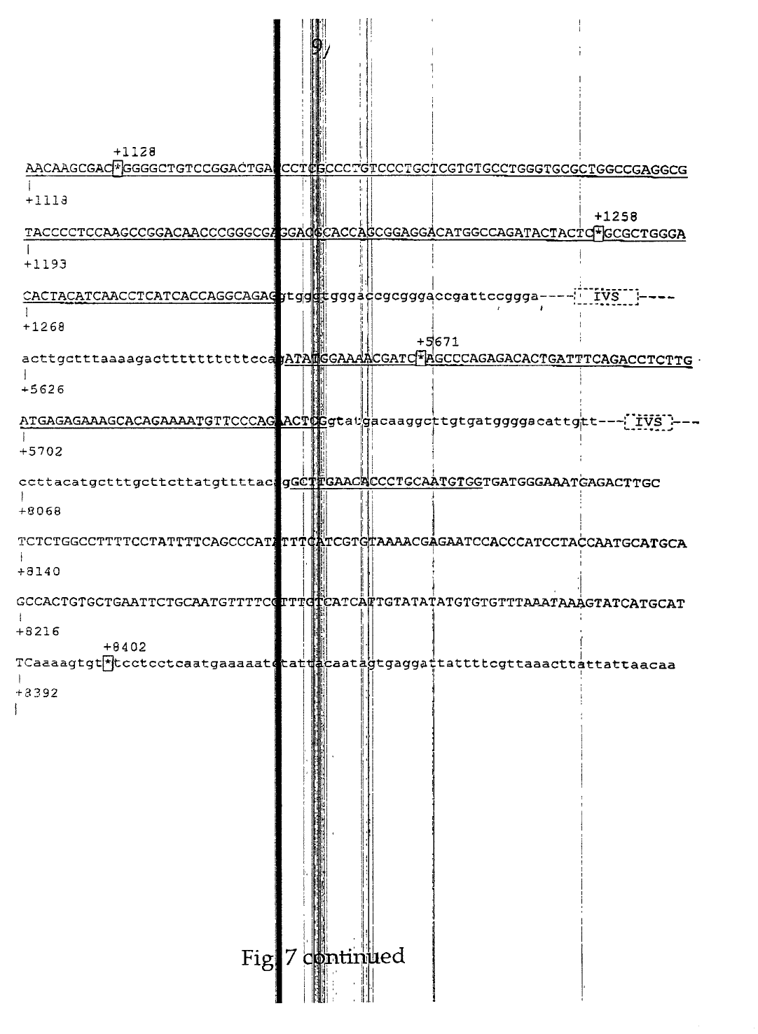

(b) analyzing said nucleic acid molecule to detect a genetic polymorphism in the human neuropeptide Y gene at a position defined as position 1128 in FIG. 7. The said position is also shown as position 106 in SEQ ID NO: I and position 49 in SEQ ID NO: 4.

In particular, the said polymorphism is the single nucleotide polymorphism 1128T/C resulting in the substitution of leucine by proline at residue 7 in the signal peptide part of pre-pro-neuropeptide Y (SEQ ID NO: 2).

The said predisposition for obesity can preferably be determined as a genetic susceptibility for increased body-mass index (BMI) defined as weight in kilograms divided by square of the height in meters. The terms “predisposition” or “susceptibility” or refer to the likelihood that an individual will develop a particular disease, condition or disorder. A subject with an increased predisposition or susceptibility will thus be more likely than average to develop a disease.

In a further aspect, the invention provides a method for diagnosis of one or more single nucleotide polymorphisms in the neuropeptide Y gene in a human individual, comprising determining the sequence of the nucleic acid of the said human individual at one or more positions as defined in FIG. 7, said positions selected from: −602;−399;−84; 1008; 1057; and 9402 The said polymorphism is preferably located in the promoter region of the human, neuropeptide Y gene, i.e. located in position −602, −399, or −84.

The method can be used in assessing the predisposition of an individual to a medical condition mediated by neuropeptide Y, such as obesity, for instance obesity determined by an increased body-mass index.

In yet another aspect, the invention provides a nucleic acid molecule comprising at least 10 contiguous nucleotides of the sequence shown in FIG. 7, having

T at position −602;

T at position −399;

C at position −84;

T at position 1008;

G at position 1057; and/or

G at position 8402.

Throughout this description the terms “standard protocols” and “standard procedures”, when used in the context of molecular biology techniques, are to be understood as protocols and procedures found in an ordinary laboratory manual such as: Current Protocols in Molecular Biology, editors F. Ausubel et al., John Wiley and Sons, Inc. 1994, or Sambrook. J., Fritsch, E. F. and Maniatis, T., Molecular Cloning: A laboratory manual, 2nd Ed., Cold Spring Harbor Laboratory Press, Cold Spring Harbor, NY 1989.

### EXAMPLES

### Example 1;

### Identification of polymorphisms

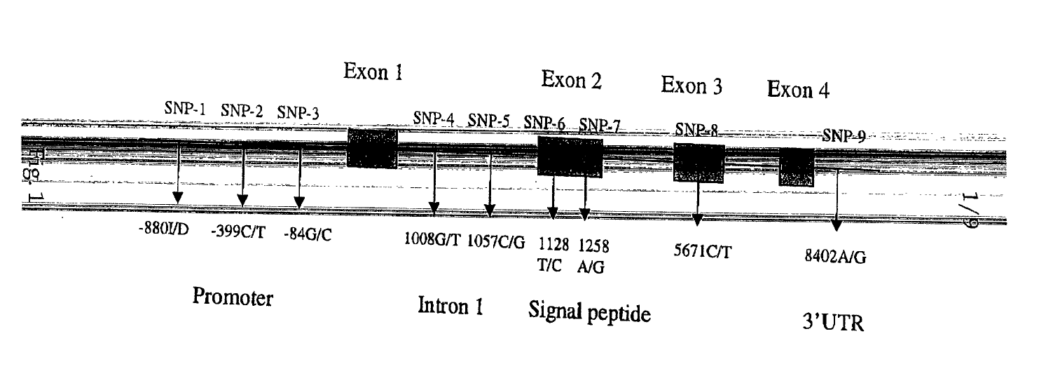

Using direct PCR product sequencing methods, we sequenced the whole NPY gene from 30 randomly selected Swedish healthy subjects. A total of 1649 bp of the 9.6 kb sequence was read in each individual. Eight single-nucleotide polymorphisms (SNPs) were found (FIG. 1). This yielded an average of I SNP/183 nucleotides sequenced. The polymorphisms consisted of 5 transitions (5 of 8, 62.5%) and 3 transversions (3 of 8, 37.5%). Transitions were more frequent than transversions as expected. These SNPs contain two variants in promoter region (SNP-2 aid -3) from the start site, synonymous base pair changes within exon 2 (SNP-7) and exon 3 (SNP-8 ), and three single base pair substitution within intron 1(SNP-4 and -5) and 3′ untranslated region (LTTR) (SNP-9). One nonsynonymous SNP (SNP-6, L7/P) was found in the signal peptide part of pre-pro-NPY within exon 2.

FIG. 1 shows a schematic of human NPY gene polymorphisms in the Swedish population. The 9.6 kb of the NPY locus is illustrated One ˜1.3 kb and three ˜500bp regions were amplified by PCR 30 unrelated individuals. These regions were sequenced and found to contain 8 SNPs. UTR, untranslated region. The SNP positions are based on the nucleotide sequence of the human NPY identified by Minth et al. (3) and on the GenBank sequence (accession no. AC004485). But the GenBank sequence should be read as complementary and reverse direction to be corresponding to that by Minth et al. SNP-1(−8801/D) stands for two base pair TG insertion (I) or deletion (C), data obtained from (M. S. Bray, B. Boerwinkle, C. L. Hanis, Obes Res. 8,219 (2000)).

### Example 2

### Genotyping of Netropeptide Y SNPs

We first analyzed the effect of nine SNPs on obesity and metabolic parameters

The POLCA individuals (n=572) were from the greater Stockholm area using a registry containing all permanent resident (Swedish origin) in the Stockholm region They are all 50 years old men without a history of cardiovascular disease, severely impaired renal function., arthritis, collagenosis, diabetes mellitus, a history of alcohol abuse or other forms of addiction.

The OBESITY population (n=674 consisted of adult women who were either healthy control subjects (n=398), with varying BMI ((BMI<30 kg/m², no medical history) or referred to the hospital's unit for uncomplicated hernia, gallstone or gastric binding for uncomplicated obesity. All individuals were from communities nearby Huddinge University Hospital, Sweden.

SNPs were genotyped by Dynastic Allele-Specific Hybridization (DASH) (Howell et al. (1999) Nature Biotechnol. 17: 87-88) in which 5 ng of each genomic DNA was amplified by PCR; 10 μl of each PCR product was transferred to a streptavidin-coated microtiter plate. Allele-specific probes were hybridized to the PCR products according to an optimized protocol (Howell et al., supra).

Differences in BMI according to genotypes were tested using analysis of variance (ANOVA) for repeated measurements using the StatView 5.0 program (SAS Institute Inc., Cary, USA). Statistical significance was accepted at p≦0.05.

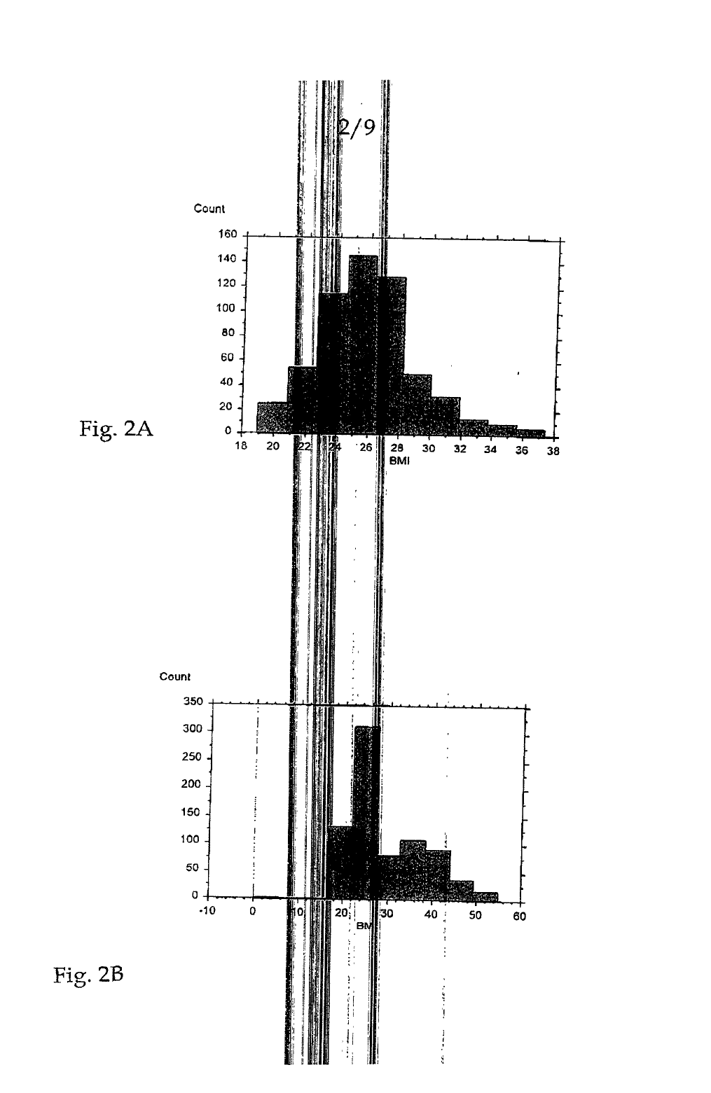

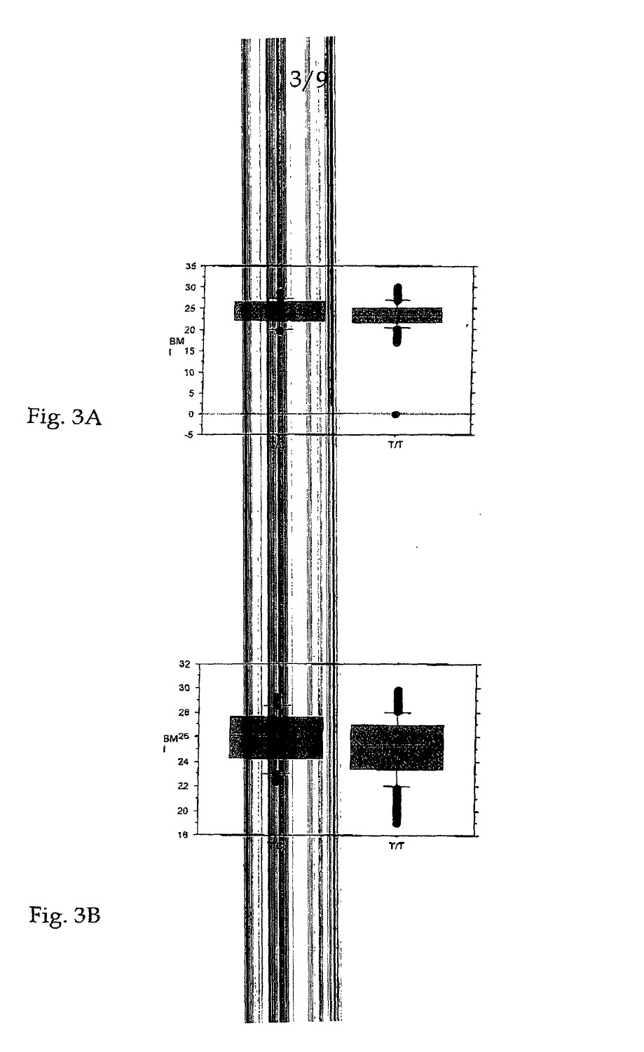

We found association of SNP-6 with BMI (Table 1). No individual with genotype CC was found in population 1. In the entire POLCA population (FIG. 2A) subjects with allele 1128C had higher mean BMI values (26.9±2.8 compared with 25.9±3.2, P=0.039, ANOVA) in the entire study sample (n=572), (FIG. 3A). When the data were analyzed separately by obesity status, in all individuals with BMI under 30 kg/² (n=510), subjectes with 128C also, had higher mean BMI values (25.9±2.0 compared with 25.1 ±2.4, P=0.043), but no significant association was found in individuals with BMI above 30 kg/m²(n=62). Serum triglyceride, serum cholesterol, VLDL cholesterol, LDL-cholesterol, systolic blood pressure and diastolic blood pressure did not differ between the SNP-6 genotype groups in total subjects or obese and non-obese subjects The remaining 8 SNPs did not show any association to BMI, serum triglyceride, serum cholesterol, VLDL cholesterol, LDL-cholesterol, systolic blood pressure and diastolic blood pressure. To make sure our genotype data are reliable, we further used RFLP to double check our result (for SNP-6 only). Both DASH and RFLP matched perfectly.

In the OBESITY population (FIG. 2B), a significant association was found in non-obese individuals (BMI below 30 kg/m²) subjects with the 1128C(Pro(7)) allele showed a mean BMI value of 24.538±2.735. compared with 23.227±3.027 for individuals without the 1128C(Pro(7)) allele (p=0.0202) (FIG. 3B). No significant association between BMI and the 1128C allele was found in individuals with BMI above 30kg/m² or in the entire OBESITY population.

To confirm our result, a second population (population 2) consist of case (BMI>30 kg/m²n=278) and control (BMI<30 kg/m² :n=396) were further genotyped for SNP-6 (Table 1). A significant association wad: found in non-obese individuals (n=396, p=0.020), but no significant association was found in individuals with BMI above 30kg/m²(n=278) and the entire study samples (n=674).

There was no deviation from Hardy-Weinberg equilibrium for the SNP-6 alleles in both populations. There was no significant difference in allele frequencies and genotype distribution among obese and non-obese individuals in the two populations.

### Example 3

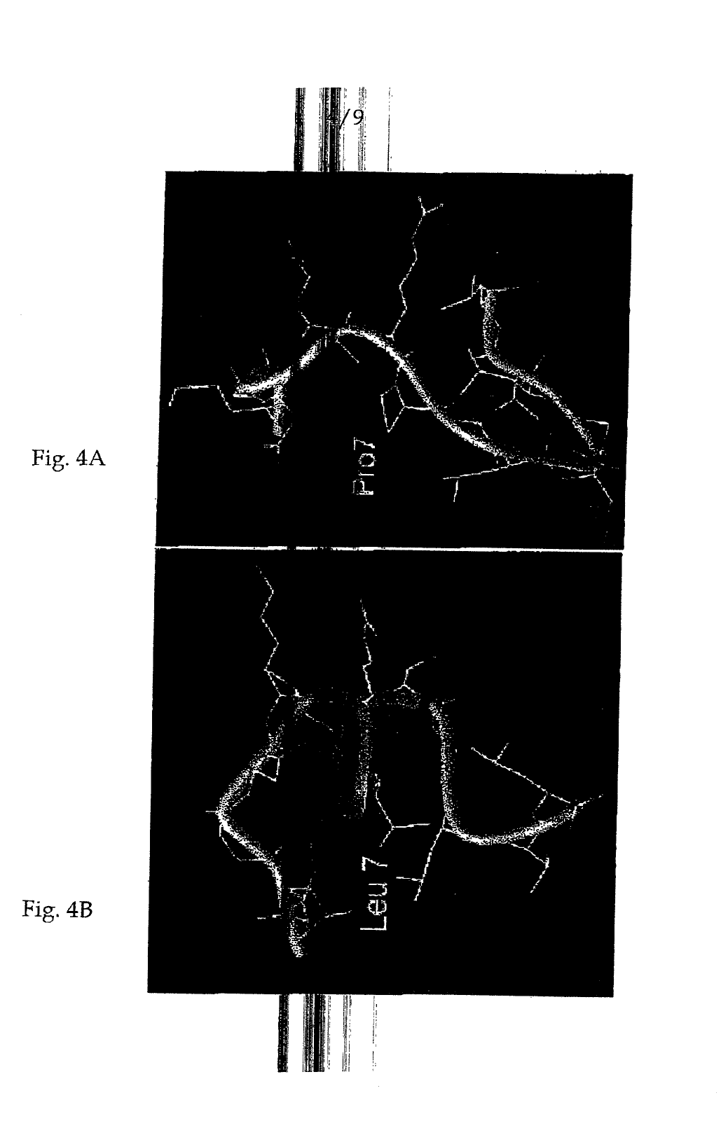

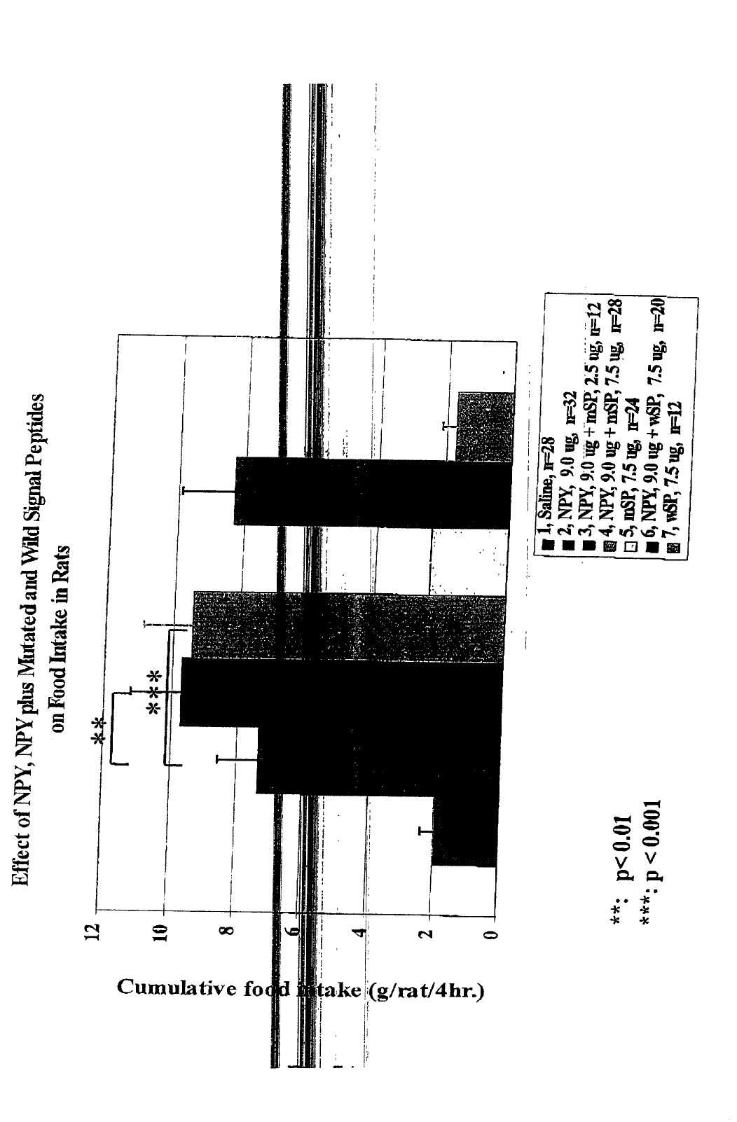

In an attempt to analyse the three dimensional structure of the two signal peptides, prediction of the peptide folding for 14 residues out of 28 (wild type MLGNKRLLSGLTL and in Leu7Pro MLGNKRPGLSGLTL) was performed using the BPMC computer modelling simulation system. Although peptide structure prediction methods by default contains a degree of uncertainty, the results indicate that it is likely that the native NPY signal peptide has clear helix propensity properties (FIG. 4A, 4B). The introduction of proline at position 7 disrupts the local conformation of the peptide by altering the packing of the helical bundle and diminishes helix propensity for the sequence. Thus, the introduction of proline into the predicted helical feature of the NPY signal peptide is likely to affect the likeliness to correctly interact with protein components essential for sub-cellular trafficking

FIGS. 4A and B are proposed models of the three dimensional structure of (a) wild type signal peptide residues 1-14 (MLGNKRLGLSGLTL) and (b) the Leu7Pro signal peptide residues 1-14 (MLGNKRPGLSGLTL). The models were created using the biased probability Monte Carlo (BPMC) algorithm (Abagyan and Totrov, 1994).

Prediction of the three dimensional peptide structures of the NPY signal peptides so (residues 1-14) was performed using The bias probability Monte Carlo (BPMC) algorithm (R. A. Abagyan, M. M. Totrov, D. N. Kuznetsov, J. Comput Chem. 15, 488 (1994)) delivered by ICM Molsoft LLC (26), http://www.molsoft.com/) The simulation contained calculations of 20 million energy evaluations with local minimization. The MIMEL (S. V. Evans, J. Mol. Graphics Model. 11, 134(1993)) method for treating electrostatic interactions in solvated systems was applied. The conformation of the lowest energy minimum was taken as a model of the fold of the peptide in solution. The resulting models were analysed and visualized on a Silicon Graphics workstation, using the Sybyl 6.3 program package (TRIPOS Associates, 1996) and the Setor program (S. V. Evans, J. Mol. Graphics Model. 11, 134(1993)).

### EXAMPLE4

To investigate any function change caused by the leucine(7)-to-proline(7) polymorphism for endoplasmic reticulum (ER) translocation, we conducted an in vitro expression experiment. The expression product with glycosylation and cleaved fragment could be clearly seen from the FIG. In the lanes with acceptor peptide (NLT) the glycosylation is reduce giving rise to a cleaved, unglycosylated lower band and a cleaved glycosylated higher band. However, no difference was found between wide-type and mutants. Our results suggest that the mutation does not affect the site of cleavage and targeting or uptake of NPY into the ER.

The wide-type signal peptide (SP),SP-NPY and SP-NPY-Cpon were for this experiment cloned into the EGFP-N3 vector (Clontech) by conventional techniques at the EcoRI and BamHI sites. Mutants were generated using the mutagenic oligonucleotides: mutated nucleotides are indicated in parentheses Forward strand, 5.GGAATTCACCATGCTAGGTAACAAGCGCC(C)GGGGCTGTCCGGA-3′; reverse strand 5′-CGGGATCCCGCCTCGGCCAGGCACC-3′ for mut-sp; Forward strand, 5′-GGAATTCACCATGCTAGGTAACAAGCGCC(C)GGGGCTGTCCGGA-3′, reverse strand, 5′-CGGGATCCATATCTCTGCCTGGTGAT-3′ for mut-SP-NPY; Forward strand, 5-′GGAATTCACCATGCTAGGTAACAAGCGCC(C)GGGGCTGTCCGGA-3′, reverse strand 5′-CGGGATCCCCACATTGCAGGGTCTTC-3′ For mut-SP-NPY-Cpon. All wide-type and mutants generated were completely sequenced in both directions to confirm the presence of the target sequence and to rule out any additional undesired changes.

PC12 cells were cultured in DMEM with 10% fetal calf serum (FCS), 5% horse serum, 100 1 U/ml penicillin and 0.1 mg/ml streptomycin. GFP was attacted to the C-terminus of prepro-NPY by subconing the human NPY cDNA including signal eptide and C-terminal peptide into a pEGFP-N3 vector (Clontech). The Leucine 7 was changed to Proline by PCR PC12 cells were transfected using Lipefectamine 2000 Reagent (GibcoBRL) and 24 h later cells were fixed with 3.7% paraformaldehyde and mounted. GFP fluorescence was visualised in a Zeiss LSM510 confocal microscope.

### Conclusion

There was no difference in the distribution of GFP between wide type and mutant for the sp-NPY-Cpon-GFP and sp-NPY-GFP constructs. GFP fluorescence was detected in the Golgi and in dots in the cytoplasm, which might correspond to vesicles. In contrast, a marked difference was seen when only the signal peptide was fused to GFP. The mutant signal peptide caused GFP to localise more diffusly in the cell.

### Example 5

### In Vitro Expression

To investigate any function change caused by the leucine(7)-to-proline(7) polymorphism for endoplasmic reticulum (ER) translocation, an in vitro expression experiment was performed.

XbaI and NdeI restriction sites were introduced by PCR at the 5′- and 3′-ends of the human NPY cDNA. Site-directed mutagenesis was performed to mutate one single nucleotide (T→C, Leu7→Pro mutation) at the second position in codon 7 (corresponding to position 106 in SEQ ID NO. 1) by PCR amplification. Two mutants with signal peptide sequence and entire NPY cDNA were produced, respectively. The XbaI and NdeI restricted PCR fragments we cloned into a pGEMI-derived vector containing the P2 domain (codon 81-323) of Escherichia coli protein leader peptidase (Lep) preceded by an NdeI site. The constructs in pGEM1 were transcribed by SP6 RNA polymerase for 1 h at 37° C. in a transcription mixture composed of 1-5 μg DNA template, 5 μL 10×SP6 H-buffer (400 mM Hepes/KOH pH 7.4, 60 mM magnesium acetate, 20 mM spermidine hydrochloride), 5 μL BSA (1 mg.mL-1), 5 μL m7G(5¢)ppp(5¢) G (10 mM). 5 μL;dithiothreitol (50 mM), 5 μL gNTP mix (10 mM ATP, 10 mM CTP, 10 mM UTP, 5 GTP), 18.5 μL water, 1.5 μL RNase inhibitor (50 units) and 0.5 μL SP6 RNA polymerase (20 units). Translation was performed in reticulocyte lysate in the presence of dog pancreas microsomes. The translation products were analyzed by SDS-PAGE.

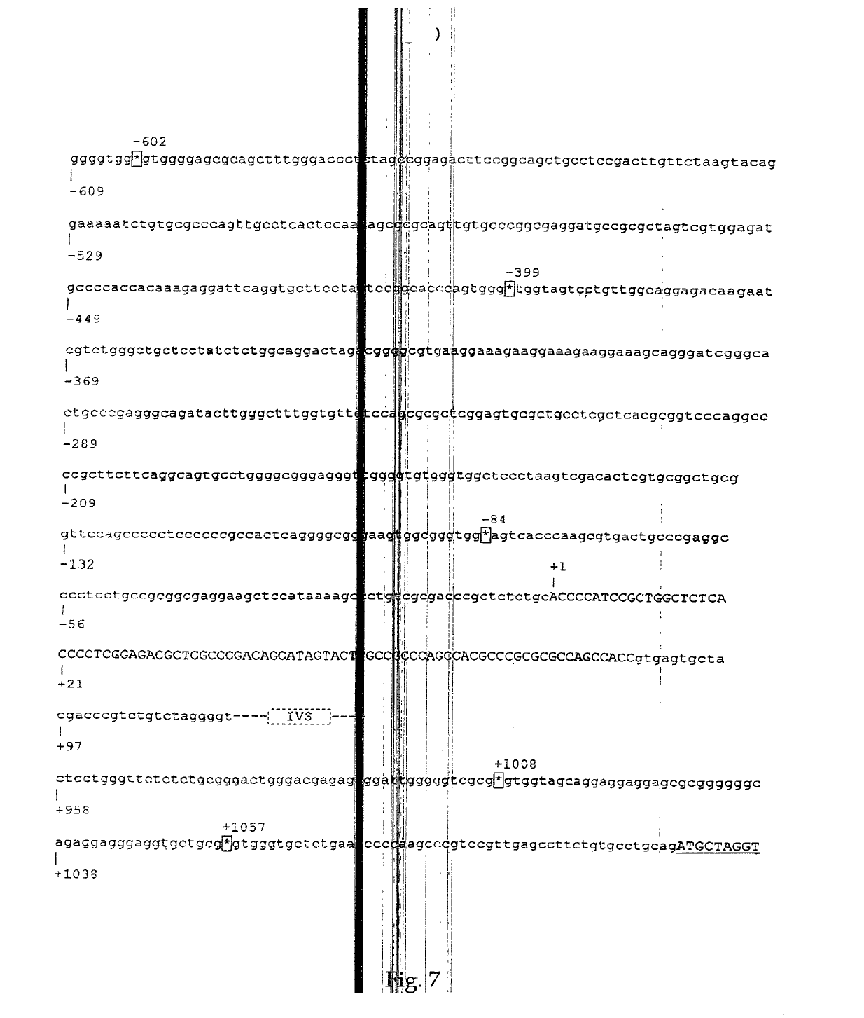

The expression products are shown as dark bands in FIG. 6. In the lanes where the acceptor peptide (NLT) is included, glycosylation is reduced, giving rise to a cleaved, unglycosylated lower band and a cleaved glycosylated higher band. However, no difference was found between wild-type an mutants. These results suggest that the mutation does not affect the site of cleavage and targeting or uptake of NPY into the ER.

### Example 6

Male Sprague-Dawley rats ranging from 280 to 320 grams were stereotaxically implanted with a guide cannula at following coordinates taken from bregma according to the atlas of Franklin and Paxinons AP: −1.0 mm, ML: 1.3 mm and DV: 4.0 mm. The rats were individually housed in cages for a week recovery after surgery. Seven groups of rats with various number of rats were divided for saline and NPY and/or plus two types of signal peptides. When monitored food intake, Rats were individually housed in polypropylene cages with metal grid floors. Animals had free access to a standard rat diet and tap water at all times and rats at a temperature of 21 +/−1° C. and 60% of humidity. Animals with good conditions had three to four days washout and then divided into groups at random for reusing in &feeding experiments. On the study day, animals randomly allocated to each group. Rats were injected brain intracerebroventricular (i.c.v.) with saline, 9 ug of mammalian neuropeptide Y and 2.5 and 7.5 ug of mutated and wild signal peptides alone and/or co-injected with wild or mutated signal peptides. Food intake was monitored after i.c.v. injection. Food spillage in the button of cage and food left in the cage were collected and weighed at the time of 30 minutes, one hour, two hours and four hour.

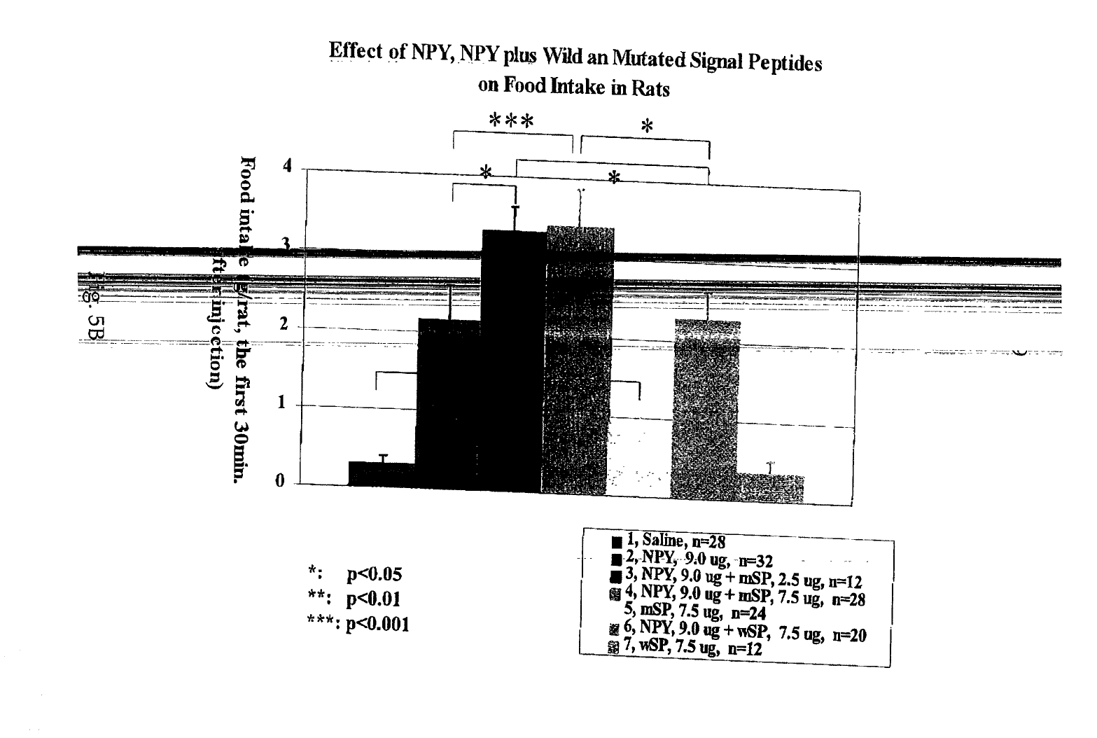

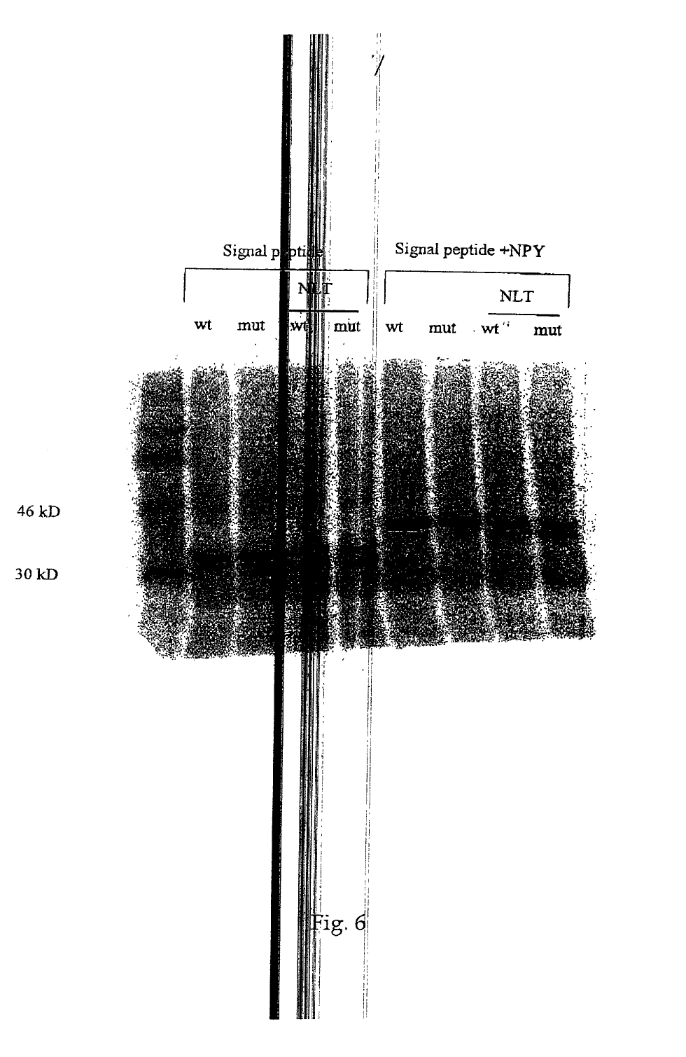

These in vivo animal studies show that co-icv administration of NPY and mutated signal peptide at two doses (2.5 and 7.5,ug) can significantly elevate overall food intake in the four hours period. In particular, after the signal icv injection 30 minutes, food intake in these two groups (3 and 4), was markedly higher than that in the group 2, treated with NPY alone and group 6, treated with NPY plus wild signal peptide (FIG. 5A). Co-icv injection high dose of mutated signal peptide (7.5 ug/rat) seems not to be able to significantly increase overall food intake, compared to that at dose of 2.5 ug/rat. On the other hand. NPY plus wild-type signal peptide can not show an increase in overall food intake. Overall cumulative food intake in the combination of NPY and mutated signal peptide also was significantly increased, compared to that in the group treated with NPY alone. However, cumulative food intake in the groups of NPY plus mutated signal peptide was not significant elevated in four hours period, compared to the group treated with NPY plus wild peptide. This is may implies that mutated signal peptide rapidly metabolized in the central nervous system Interestingly, mutated signal peptide alone injected was found to elevate food intake in the first 30 minutes after injection (FIG. 5B), which suggests that the signal peptide may modulate food intake However, the mutated peptide was not able to induce increase in cumulative food intake in four hours period.

In FIG. 5A the effect of single co-injection of NPY (9 ug/rat) with and without mutated (2.5 and 7.5 ug/rat) and wild signal peptides (7.5 ug/rat) on food intake post injection of the peptides, 4 hours period, Data are expressed as mean+/−SEM. Number of rats in each group is indicated. *p<0.05;, p<0.01 and ***p <0.001 vs the corresponding NPY alone or NPY plus wild signal peptide injected. In FIG. 5A the effect of single co-injection of NPY (9 ug/rat) with and without mutated (2.5 and 7.5 ug/rat) and Wild signal peptides (7.5 ug/rat) on food intake, the first 30 minutes period after icv injection, all other is the same as FIG. 5A.

As seen from the appendix FIG. 5 we can thus now offer an explanation why it is that the 1128 polymorphism (which gives rise to a amino acid change, leucine to proline) causes increased body mass index, (BMI). The amino acid change gives rise to an altered signal peptide that in itself is able to augment the stimulatory effect of neuropeptide Y (NPY) on food intake in rats. This phenomenon can occur in humans and therefore account for the increase in BMI indicator of obesity) that is seen in humans with this polymorphism.

TABLE 1
Mean values for BMI in two non-obese Swedish populations

| | Population 1 + Population 2 | | | | | | | | | | | |
|---|---|---|---|---|---|---|---|---|---|---|---|---|
| | (N = 906) | | | | Population 1 (N = 510) | | | | Population 2 (N = 396) | | | |
| | TT | TC | CC | p | TT | TC | CC | P | TT | TC | CC | P |
| Total | 24.510 ± | 25.349 ± | — | 0.023 | 25.122 ± | 25.935 ± | — | 0.043 | 23.227 ± | 24.538 ± | — | 0.020 |
| | 2.597 | 2.330 | | | 2.376 | 1.984 | | | 3.027 | 2.735 | | |

1. A method for diagnosing predisposition for obesity in a human individual, comprising
(a) obtaining a biological sample containing at least one nucleic acid molecule from said human individual; and
(b) analyzing said nucleic acid molecule to detect a genetic polymorphism in the human neuropeptide Y gene at a position at defined as position 1128 FIG. 7.

2. The method according to claim 1 wherein said polymorphism results in the substitution of leucine by proline at residue 7 in the signal peptide part of pre-pro-neuropeptide Y.

3. The method according to claim 1 or 2 when predisposition for obesity is determined as a genetic susceptibility for increased body-mass index.

4. A method for diagnosis of one or more single nucleotide polymorphisms in the neuropeptide Y gene in a human individual, comprising determining the sequence of the nucleic acid of the said human individual at one or more positions as defined in FIG. 7, said positions selected from:
−602;
−399;
−84;
1008;
1057; and
8402.

5. The method according to claim 4 for use in assessing the predisposition of an individual to a medical condition mediated by neuropeptide Y.

6. The method according to claim 5 wherein said medical condition is obesity.

7. The method according to claim 6 wherein obesity is determined by an increased body-mass index.

8. The method according to an one of claims 4 to 6 wherein the said polymorphism is in position −602, −399, or −84 in the promoter region of the human neuropeptide Y gene.

9. A nucleic acid molecule comprising at least 10 contiguous nucleotides of the sequence shown in FIG. 7 having
T at position −602;
T at position −399;
C at position −84;
T at position 1 008;
G at position 1057; and/or
G at position 8402.


SEQUENCE LISTING (6 sequences)

SEQ ID NO: 1 - DNA, 551 bp, human
  CDS (87)..(380)
accccatccgctggctctcacccctcggagacgctcgcccgacagcatagtacttgccgcccagccacgcccgcgcgccagccaccatgctaggtaacaagcgactggggctgtccggactgaccctcgccctgtccctgctcgtgtgcctgggtgcgctggccgaggcgtacccctccaagccggacaacccgggcgaggacgcaccagcggaggacatggccagatactactcggcgctgcgacactacatcaacctcatcaccaggcagagatatggaaaacgatccagcccagagacactgatttcagacctcttgatgagagaaagcacagaaaatgttcccagaactcggcttgaagaccctgcaatgtggtgatgggaaatgagacttgctctctggccttttcctattttcagcccatatttcatcgtgtaaaacgagaatccacccatcctaccaatgcatgcagccactgtgctgaattctgcaatgttttcctttgtcatcattgtatatatgtgtgtttaaataaagtatcatgcattc

SEQ ID NO: 2 - protein, 97 aa, human
MLGNKRLGLSGLTLALSLLVCLGALAEAYPSKPDNPGEDAPAEDMARYYSALRHYINLITRQRYGKRSSPETLISDLLMRESTENVPRTRLEDPAMW

SEQ ID NO: 3 - DNA, 325 bp, human
  prim_transcript (210)..(325)
ccgcttcttcaggcagtgcctggggcgggagggttggggtgtgggtggctccctaagtcgacactcgtgcggctgcggttccagccccctccccccgccactcaggggcgggaagtggcgggtgggagtcacccaagcgtgactgcccgaggcccctcctgccgcggcgaggaagctccataaaagccctgtcgcgacccgctctctgcaccccatccgctggctctcacccctcggagacgctcgcccgacagcatagtacttgccgcccagccacgcccgcgcgccagccaccgtgagtgctacgacccgtctgtctaggggt

SEQ ID NO: 4 - DNA, 247 bp, human
  Intron (1)..(29)
cccgtccgttgagccttctgtgcctgcagatgctaggtaacaagcgactggggctgtccggactgaccctcgccctgtccctgctcgtgtgcctgggtgcgctggccgaggcgtacccctccaagccggacaacccgggcgaggacgcaccagcggaggacatggccagatactactcagcgctgggacactacatcaacctcatcaccaggcagaggtgggtgggaccgcgggaccgattccggga

SEQ ID NO: 5 - DNA, 142 bp, human
  Intron (1)..(30)
acttgctttaaaagacttttttttttccagatatggaaaacgatctagcccagagacactgatttcagacctcttgatgagagaaagcacagaaaatgttcccagaactcggtatgacaaggcttgtgatggggacattgtt

SEQ ID NO: 6 - DNA, 300 bp, human
  Intron (1)..(31)
ccttacatgctttgcttcttatgttttacaggcttgaagaccctgcaatgtggtgatgggaaatgagacttgctctctggccttttcctattttcagcccatatttcatcgtgtaaaacgagaatccacccatcctaccaatgcatgcagccactgtgctgaattctgcaatgttttcctttgtcatcattgtatatatgtgtgtttaaataaagtatcatgcattcaaaagtgtatcctcctcaatgaaaaatctattacaatagtgaggattattttcgttaaacttattattaacaa